(19) United States
(12) Patent Application Publication
(10) Pub. No.: US 2006/0234321 A1
(43) Pub. Date: Oct. 19, 2006

(54) Syphilis diagnostic tests and kits

(75) Inventors: Wesley C. Van Voorhis, Seattle, WA (US); Caroline Elizabeth Cameron, Seattle, WA (US); Sheila Ann Lukehart, Seattle, WA (US); Janelle Mei Ying Kuroiwa, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: University of Washington, Seattle, WA (US)

(21) Appl. No.: 11/107,373

(22) Filed: Apr. 14, 2005

(51) Int. Cl. G01N 33/554; G01N 33/569

(52) U.S. Cl. 435/7.32; 435/7.34

(57) ABSTRACT

The present invention provides methods and kits for determining whether a human subject is infected with T. pallidum, and isolated fragments of T. pallidum protein Tp92 that can be used in the methods and kits of the invention.

### STATEMENT OF GOVERNMENT LICENSE RIGHTS

The U.S. Government has a paid-up license in this invention and the right in limited circumstances to require the patent owner to license others on reasonable terms as provided for by the terms of Grant No. R01 A1-43456 awarded by the National Institutes of Health, and Grant No. R01 A1-51334 awarded by the National Institutes of Health.

### FIELD OF THE INVENTION

The present invention relates to methods for diagnosing syphilis in human beings.

### BACKGROUND OF THE INVENTION

The spirochaetes are a phylum of distinctive bacteria which have long, helically coiled, cells. They are distinguished by the presence of flagella, called axial filaments, that run lengthwise between the cell membrane and cell wall. These cause a twisting motion which allows the spirochaete to move about. Most spirochaetes are free-living and anaerobic.

Treponema pallidum, subspecies pallidum, (hereinafter referred to as Treponema pallidum or T. pallidum) is a spirochaete that causes syphilis in human beings. T. pallidum enters the host via breaches in squamous or columnar epithelium, and primary infection is normally via sexual contact. T. pallidum can be transmitted to a fetus by transplacental passage during the later stages of pregnancy.

Syphilis is characterized by primary, secondary and tertiary clinical stages. The primary stage involves multiplication of the bacteria at the site of entry to produce a localized infection. The secondary stage occurs following an asymptomatic period and involves dissemination of the bacteria to other tissues. The tertiary stage may not occur for many years after infection, and can cause damage to the brain and central nervous system, and ultimately lead to death.

Syphilis diagnosis during the early primary stage can be accomplished by dark-field microscopy of a sample of primary chancre to identify the presence of spirochetes. Following the resolution of the primary chancre, and in clinics lacking dark-field microscopy, the mainstay of syphilis diagnosis is a variety of serologic tests. The most common screening tests are the rapid plasma reagin (RPR) and Venereal Disease Research Laboratory (VDRL) tests, both of which test for the presence of antilipoidal antibodies. The antilipoidal antibodies recognize lipid material released from damaged host cells, and from T. pallidum. Because neither of these tests use syphilis-specific antibodies, there are problems associated with both their specificity and their sensitivity. In early primary disease antilipoidal antibodies may not have developed, and in late syphilis up to thirty percent of individuals may lack antilipoidal antibodies. In addition, because a variety of conditions (e.g., lupus and increased age) lead to antilipoidal antibodies and false-positive results, a confirmatory test is often required.

Confirmatory tests include FTA-Abs (fluorescent treponemal antibody absorption test), MHA-TP (micro-hemagglutination assay for T. pallidum), and TPHA (T. pallidum hemagglutination assay), which use crude T. pallidum antigens (Larsen, S. A., et al., J. Clin. Microbiol. 14:441-445, 1981); tests using whole T. pallidum antigen extracts; and a variety of T. pallidum recombinant protein tests (Gerber, A., et al., Immunobiology 196:535-549, 1996; Hagedorn, H. J., et al., J. Clin. Microbiol. 40:973-978, 2002; Ijsselmuiden, O. E., et al., Eur. J. Clin. Microbiol. Infect. Dis. 8:716-721, 1989; Ijsselmuiden, O. E., et al., J. Clin. Microbiol. 27:152-157, 1989; Larsen, S. A., et al., J. Clin. Microbiol. 14:441-445, 1981; Peterson, K. M., et al., J. Exp. Med., 164:1160-1170, 1986; Radolf, J. D., et al., J. Infect. Dis., 153:1023-1027, 1986; Rodriguez, I., et al., Mem. Inst. Oswaldo Cruz 97:347-349, 2002; Sambri, V., et al., Clin. Microbiol. Infect. 7:200-205, 2001; Sato, N. S., et al., Rev. Inst. Med. Trop. Sao Paulo 41:115-118, 1999; Schmidt, B. L., et al., J. Clin. Microbiol. 38:1279-1282, 2000; Schouls, L. M., et al., Infect. Immun. 57:2612-2623, 1989; Young, H., Dermatol. Clin. 16:691-698, 1998; Young, H. et al., Int. J. STD AIDS 11:288-291, 2000; Young, H, et al., Int. J. STD AIDS 9:196-200, 1998; Young, H., et al., J. Clin. Microbiol. 36:913-917; Zrein, M., et al., J. Clin. Microbiol. 33:525-527).

Improved syphilis diagnostic tests are, nonetheless, required because the RPR and VDRL tests give false positives, require a secondary specific test, and are not sensitive in detecting early syphilis. Moreover, many, if not all, of the available recombinant Treponema proteins, that could, in principle, be used in a syphilis diagnostic test, do not react with antibodies from syphilitic individuals with sufficient specificity.

### SUMMARY OF THE INVENTION

In accordance with the foregoing, the present inventors have identified T. pallidum proteins, and fragments thereof, that can be used, alone or in combination, in syphilis diagnostic tests. The T. pallidum proteins are of three types: Tp92 proteins, Tp0453 proteins and Gpd proteins. Each of the foregoing three types of proteins are believed to be located, in vivo, in the outer membrane of T. pallidum, which may explain their immunogenicity. SEQ ID NO:1 sets forth the nucleic acid sequence of a gene encoding the representative Tp92 protein having the amino acid sequence set forth in SEQ ID NO:2. SEQ ID NO:3 sets forth the nucleic acid sequence of a gene encoding the representative Tp0453 protein having the amino acid sequence set forth in SEQ ID NO:4. SEQ ID NO:5 sets forth the nucleic acid sequence of a gene encoding the representative Gpd protein having the amino acid sequence set forth in SEQ ID NO:6.

Thus, in one aspect, the present invention provides methods for determining whether a human subject is infected with T. pallidum. The methods of this aspect of the invention each include the steps of: (a) contacting an isolated polypeptide selected from the group consisting of a fragment of at least twelve consecutive amino acids of a Tp92 protein, a fragment of at least twelve consecutive amino acids of a Tp0453 protein, and a fragment of at least twelve consecutive amino acids of a Gpd protein, with antibodies from a human subject; and (b) determining whether the antibodies bind to the isolated polypeptide, wherein binding of the antibodies to the isolated polypeptide indicates that the human subject is infected with T. pallidum.

In another aspect, the present invention provides kits that each include: (a) a substrate comprising an isolated polypeptide selected from the group consisting of a fragment of at least twelve consecutive amino acids of a Tp92 protein, a fragment of at least twelve consecutive amino acids of a Tp0453 protein, and a fragment of at least twelve consecutive amino acids of a Gpd protein; (b) reagents for labeling antibody bound to the isolated polypeptide; and (c) written indicia providing a user with instructions for use of the kit to determine whether a human subject is infected with T. pallidum. The kits can be used, for example, in the practice of the methods of the invention for determining whether a human subject is infected with T. pallidum.

In another aspect, the present invention provides the isolated polypeptides having the amino acid sequences set forth in SEQ ID NOS:7-21, which are fragments of the Tp92 polypeptide having the amino acid sequence set forth in SEQ ID NO:2. Thus, in one aspect, the present invention provides isolated polypeptides including one or more amino acid sequence(s) selected from the group consisting of SEQ ID NOS:7-21. The isolated polypeptides of the invention can be used, for example, in the methods and kits of the invention.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a bar chart showing the reactivity of a panel of sera from human syphilis patients against Tp92 (SEQ ID NO:2) and 15 fragments of Tp92 (SEQ ID NOS:7-21). Shaded bars represent the reactivity of human serum from syphilis patients. Unshaded bars represent the reactivity of human serum from subjects who were not infected with T. pallidum.

FIG. 2 shows a bar chart showing the reactivity of a panel of sera from rabbits (artificially infected with T. pallidum) against Tp92 (SEQ ID NO:2) and 15 fragments of Tp92 (SEQ ID NOS:7-21). Shaded bars represent the reactivity of serum from rabbits infected with T. pallidum. Unshaded bars represent the reactivity of serum from rabbits that were not infected with T. pallidum.

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al. (1989) Molecular Cloning: A Laboratory Manual, 2nd ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

As used herein, the term “Tp92 protein” refers to a protein that is at least 70% identical to the Tp92 protein having the amino acid sequence set forth in SEQ ID NO:2 (having GenBank accession number AF152012).

As used herein, the term “Tp0453 protein” refers to a protein that is at least 70% identical to the Tp0453 protein having the amino acid sequence set forth in SEQ ID NO:4 (having GenBank accession number NC_{—}000919).

As used herein, the term “Gpd protein” refers to glycerophosphodiester phosphodiesterase, a protein that catalyzes the hydrolysis of glycerophosphodiesters from phospholipids and triglycerides to glycerol 3-phosphate, and that is at least 70% identical to the Gpd protein having the amino acid sequence set forth in SEQ ID NO:6 (having GenBank accession number AF127421).

As used herein, the term “T. pallidum” refers to Treponema pallidum, subspecies pallidum, the spirochaete bacterium that causes syphilis.

As used herein, the term “isolated polypeptide” refers to a polypeptide that is substantially (such as at least 99% pure) or completely free from components which normally accompany it as found in its native state. Purity and homogeneity may be determined using analytical chemistry techniques such as polyacrylamide gel electrophoresis or high performance liquid chromatography. An isolated polypeptide is typically the predominant or sole band when visualized using polyacrylamide gel electrophoresis.

The term “percent identical” or “percent identity”, or grammatical equivalents thereof, as applied to a polypeptide is the percentage of amino acid residues in a candidate polypeptide sequence that are identical with a subject polypeptide sequence, after aligning the sequences to achieve the maximum percent identity. No gaps are introduced into the candidate polypeptide sequence in order to achieve the best alignment. Amino acid sequence identity can be determined, for example, in the following manner. The subject polypeptide sequence is used to search a polypeptide sequence database, such as the GenBank database (maintained by the National Center for Biotechnology Information, National Library of Medicine, Building 38A, Bethesda, Md. 20894, U.S.A.) using the BLASTP program. The program is used in the ungapped mode. Default filtering is used to remove sequence homologies due to regions of low complexity. The default parameters of BLASTP are utilized. Filtering for sequences of low complexity utilize the SEG program. The BLASTP program identifies polypeptide sequences in the database that have a defined level of identity to the subject polypeptide sequence.

As used herein, the term “bodily fluid” refers to a liquid that is a component of the human body. Examples of bodily fluids include whole blood, blood serum, blood plasma and saliva.

In one aspect, the present invention provides methods for determining whether a human subject is infected with T. pallidum. The methods of this aspect of the invention each include the steps of: (a) contacting an isolated polypeptide selected from the group consisting of a fragment of at least twelve consecutive amino acids of a Tp92 protein, a fragment of at least twelve consecutive amino acids of a Tp0453 protein, and a fragment of at least twelve consecutive amino acids of a Gpd protein, with antibodies from a human subject; and (b) determining whether the antibodies bind to the isolated polypeptide, wherein binding of the antibodies to the isolated polypeptide indicates that the human subject is infected with T. pallidum.

In the practice of the methods of the present invention a fragment of at least twelve consecutive amino acids of a Tp92 protein, and/or a fragment of at least twelve consecutive amino acids of a Tp0453 protein, and/or a fragment of at least twelve consecutive amino acids of a Gpd protein is/are contacted with antibodies from a human subject. Typically each fragment is attached to a substrate, and the substrate is contacted with a liquid composition (such as whole blood, plasma, serum, saliva or another bodily fluid) that contains the antibodies, for a period of time sufficient to permit the antibodies to bind to the fragment(s). For example, the substrate can be completely or partially immersed in the liquid composition. Binding of the antibodies to the immobilized protein fragment(s) indicates that the human being that produced the antibodies is infected with T. pallidum.

One method of detecting the presence of antibodies bound to the polypeptide(s) is an immunoassay. One having ordinary skill in the art can readily appreciate the multitude of ways to practice an immunoassay to detect the presence of antibodies bound to the polypeptide(s). Various immunoassay procedures are described in J. Goers, “Immunochemical Techniques Laboratory Manual”, Academic Press (1993), and “Current Protocols in Immunology”, Coligan, J. E., Bierer, B. E., Margulies, D. H., Shevach, E. M., Strober, W., and Kriisbeek, A. M., eds., John Wiley and Sons Publishers, 2005 (Vols. 1-5), New York City, N.Y., which publications are incorporated herein by reference.

For example, an indirect antibody capture immunoassay (a form of enzyme-linked immunosorbent assay (ELISA)) can be used to detect the presence of antibody bound to the isolated polypeptide(s). An example of an indirect antibody capture immunoassay is described in Chapter 10, of J. Goers, “Immunochemical Techniques Laboratory Manual,” supra. For example, the wells of a plastic microtiter plate are coated with an isolated fragment of a Tp92 protein, a Tp0453 protein, or a Gpd protein, and serum from a human being is added to one or more of the coated wells. For example, serum from up to 96 different human beings (suspected of suffering from syphilis) can be added to the coated wells of a 96-well plastic microtiter plate. If the serum contains antibody that binds to one of the isolated fragments of a Tp92 protein, a Tp0453 protein, or a Gpd protein, then the antibody binds to the fragment(s) in the well and is thereafter detected by a detectably labeled molecule that selectively binds to antibodies, such as labeled protein A, or a labeled anti-class-specific antibody, or labeled anti-subclass specific antibody. A class-specific antibody specifically binds to a particular class of antibodies (e.g., IgM antibody, IgG antibody, or IgA antibody). A subclass-specific antibody specifically binds to a particular subclass of antibodies (e.g., subclasses IgG1, IgG2a, IgG2b, IgG3, or IgG4).

Examples of substrates to which the polypeptide(s) may be bound include: nitrocellulose, glass, polystyrene, polypropylene, polyethylene, dextran, nylon, amylases, natural and modified celluloses, polyacrylamides, agaroses, and magnetite. The substrate configuration may be, for example, spherical (e.g., a bead); or cylindrical (e.g., the inside surface of a test tube, or the external surface of a rod); or flat, such as a sheet, or test strip (e.g., plastic test strip). Again by way of example, polypeptide(s) may be bound to the wells of a microtiter plate. Polypeptide(s) may be covalently or non-covalently bound to a substrate.

Examples of enzymes which can be used to detectably label molecules that selectively bind to antibodies include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase.

Examples of radioactive isotopes which can be used to detectably label molecules that selectively bind to antibodies include, but are not limited to, ³H, ¹²⁵I, ¹³¹I, ³⁵S, and ¹⁴C. The radioactive isotope can be detected by such means as the use of a gamma counter or a scintillation counter or by autoradiography.

Fluorescent compounds can also be used to detectably label molecules that selectively bind to antibodies. Representative examples of useful fluorescent compounds include fluorescein isothiocyanate, rhodamine, phycoerythrin, phycocyanin, allophycocyanin, o-phthaldehyde and fluorescamine. When the fluorescent-labeled molecule is exposed to light of the proper wave length, its presence can be detected due to its fluorescence.

Again by way of example, fluorescence-emitting metals such as ¹⁵²Eu, or others of the lanthanide series, can also be used to detectably label molecules that selectively bind to antibodies. These metals can be attached using such metal chelating groups as diethylenetriaminepentaacetic acid (DTPA) or ethylenediamine-tetraacetic acid (EDTA).

Chemiluminescent compounds (e.g., luminol, isoluminol, theromatic acridinium ester, imidazole, acridinium salt and oxalate ester) and bioluminescent compounds (e.g., luciferin, luciferase and aequorin) can also be used to detectably label molecules that selectively bind to antibodies.

Molecules that selectively bind to antibodies (e.g., protein A) can also be linked to biotin, and the biotin can be detected by avidin, or streptavidin, that is detectably labeled.

As understood by those of ordinary skill in the art, positive and negative controls are typically performed in which known amounts of polypeptide and no polypeptide, respectively, are used in assays being performed in parallel with the test assay. Thus, for example, if a sample of human serum provided a positive result in a negative control, then that result would be considered artifactual.

The isolated polypeptides used in the practice of the invention are selected from the following group (1) a fragment of at least twelve consecutive amino acids of a Tp92 protein; (2) a fragment of at least twelve consecutive amino acids of a Tp0453 protein; and (3) a fragment of at least twelve consecutive amino acids of a Gpd protein. Whole Tp92 protein, and/or whole Tp0453 protein, and/or whole Gpd protein may be used in the practice of the invention.

One type of isolated polypeptide, or more than one type of isolated polypeptide, may be used in the practice of the invention. For example, the combination of a fragment of at least twelve consecutive amino acids of a Tp92 protein, a fragment of at least twelve consecutive amino acids of a Tp0453 protein and a fragment of at least twelve consecutive amino acids of a Gpd protein may be used in the practice of the invention. Again by way of example, the combination of two members of the following group may be used in the practice of the invention: a fragment of at least twelve consecutive amino acids of a Tp92 protein, a fragment of at least twelve consecutive amino acids of a Tp0453 protein and a fragment of at least twelve consecutive amino acids of a Gpd protein.

Again by way of example, a hybrid protein that includes a fragment of at least twelve consecutive amino acids from two, or three, of a Tp92 protein, a Tp0453 protein, and a Gpd protein may be used. Hybrid proteins may be made, for example, using standard nucleic acid cloning and manipulation techniques, such as the techniques described in Ausubel et al., supra. For example, to make a hybrid protein that includes a defined portion of a Tp92 protein, a defined portion of a Tp0453 protein, and a defined portion of a Gpd protein, the coding sequence for each of the foregoing portions can be excised (using restriction enzymes) from a Tp92 gene (e.g., the gene having the nucleic acid sequence set forth in SEQ ID NO:1), a Tp0453 gene (e.g., the gene having the nucleic acid sequence set forth in SEQ ID NO:3) and a Gpd gene (e.g., the gene having the nucleic acid sequence set forth in SEQ ID NO:5), respectively, and the excised portions are ligated together to form a hybrid nucleic acid molecule that is then ligated into an expression vector. The resulting, recombinant, expression vector is introduced into a suitable host cell (e.g., yeast cells) and the ericoded, hybrid, protein is expressed therein and purified from the cells. Again by way of example, the required gene portions can be amplified using PCR and then ligated together and ligated into an expression vector.

Useful expression vectors can include transcriptional and translational regulatory sequences. In general, the transcriptional and translational regulatory sequences may include, but are not limited to, promoter sequences, ribosomal binding sites, transcriptional start and stop sequences, translational start and stop sequences, and enhancer or activator sequences. Promoter sequences may be, for example, constitutive or inducible promoters. The promoters may be, for example, naturally-occurring promoters or hybrid promoters. In addition, an expression vector typically contains a selectable marker gene to allow the selection of transformed host cells. Vectors useful for expressing a desired polypeptide can be any type of vector, including plasmid vectors and viral vectors.

It is also possible to use protein synthesis techniques to synthesize a protein that includes portions of one, two, or all three of a Tp92 protein, a Tp0453 protein, and a Gpd protein. Protein synthesis techniques are described, for example, in Aimoto, S., Contemporary methods for peptide and protein synthesis. Current Organic Chemistry 5:45-87 (2001).

In another aspect, the present invention provides kits that each include: (a) a substrate comprising an isolated polypeptide selected from the group consisting of a fragment of at least twelve consecutive amino acids of a Tp92 protein, a fragment of at least twelve consecutive amino acids of a Tp0453 protein, and a fragment of at least twelve consecutive amino acids of a Gpd protein; (b) reagents for labeling antibody bound to the isolated polypeptide; and (c) written indicia providing a user with instructions for use of the kit to determine whether a human subject is infected with T. pallidum. The kits may optionally include a liquid for washing the substrate after the substrate has been contacted with a bodily fluid from a human being, and before the strip is contacted with the reagents for labeling antibody bound to the isolated polypeptide. The kits may also include packaging that contains the foregoing components.

The isolated polypeptide(s) may be covalently or non-covalently bound to the substrate. Representative examples of useful substrates are described in connection with the methods of the invention for determining whether a human subject is infected with T. pallidum. Representative reagents for labeling antibodies are also described in connection with the methods of the invention for determining whether a human subject is infected with T. pallidum. The kit also includes written indicia providing a user with instructions for use of the kit to determine whether a human subject is infected with T. pallidum. Thus, for example, the written indicia might be located upon a paper insert present within packaging that is included as part of the kit, or may be located upon part of the packaging. The written indicia may provide representative values for the amount of antibody that is bound to the isolated polypeptide(s) that indicates that the human subject is infected with T. pallidum. Additionally, the kit may optionally include depictions or photographs that represent the appearance of positive and negative results (such as a color change visible on the substrate that indicates the presence of antibodies bound to the polypeptides on the substrate).

Typically the kits of the present invention include controls, such as a substrate to which no polypeptide is bound, and a substrate that bears a polypeptide that is selected so that it is not recognized by antibodies directed against T. pallidum.

In another aspect, the present invention provides the isolated polypeptides having the amino acid sequences set forth in SEQ ID NOS:7-21, which are fragments of the Tp92 polypeptide having the amino acid sequence set forth in SEQ ID NO:2. The isolated polypeptides (SEQ ID NOS:7-21) can be used, for example, in the methods and kits of the invention. In particular, as described more fully in Example 1 herein, the isolated polypeptide having the amino acid sequence set forth in SEQ ID NO:13 is a highly immunogenic fragment of Tp92 (SEQ ID NO:2).

The following examples merely illustrate the best mode now contemplated for practicing the invention, but should not be construed to limit the invention.

### EXAMPLE 1

This Example describes the expression of the 15 Tp92 polypeptide fragments having the amino acid sequences set forth in SEQ ID NOS:7-21 in E. coli, and the purification of the 15 Tp92 polypeptide fragments (SEQ ID NOS:7-21) from E. coli. This Example also describes the results of experiments to identify which of the 15 Tp92 polypeptide fragments (SEQ ID NOS:7-21) are bound by antibodies from human syphilis patients.

Fifteen recombinant protein fragments (SEQ ID NOS:7-21) were created that span the entire length of the mature Tp92 protein (SEQ ID NO:2). Table 1 shows the boundaries of these fragments (numbering is from the first amino acid at the N-terminus of the mature Tp92 protein (SEQ ID NO:2).

TABLE 1

| Fragment | Number of First and Last | |
|---|---|---|
| Number | Amino Acid Residue | SEQ ID NO: |
| F1 | 26-592 | 7 |
| F2 | 593-837 | 8 |
| F3 | 593-759 | 9 |
| F4 | 749-837 | 10 |
| F5 | 785-837 | 11 |
| F6 | 26-764 | 12 |
| F7 | 26-220 | 13 |
| F8 | 221-592 | 14 |
| F9 | 26-123 | 15 |
| F10 | 124-220 | 16 |
| F11 | 221-344 | 17 |
| F12 | 345-456 | 18 |
| F13 | 457-592 | 19 |
| F14 | 404-775 | 20 |
| F15 | 26-403 | 21 |

The 15 Tp92 peptides (SEQ ID NOS:7-21) were prepared by subcloning PCR amplified portions of the Tp92 gene into an expression vector containing 6-histidines at the N-terminus. The recombinant vectors were transformed into E. coli, and the encoded Tp92 peptides (SEQ ID NOS:7-21) were expressed in the E. coli which were then lysed. The expressed Tp92 peptides (SEQ ID NOS:7-21) were purified from the E. coli lysates using Nickel-affinity chromatography.

Using an ELISA-based immunological detection system, a panel of serum collected from diagnosed syphilis patients were tested against these recombinant protein fragments (SEQ ID NOS:7-21), to determine where the immunological reactivity is focused within the molecule. The ELISA was carried out by coating ninety-six-well plates (Maxisorp F9; Costar) overnight at 4° C. with 50 μl of the recombinant T. pallidum proteins per well in phosphate-buffered saline (PBS), pH 7.4, with 0.1% sodium dodecyl sulfate at concentrations of 2 μg/ml of recombinant proteins and peptides. Plates were blocked at room temperature for 2 hours with 4% milk in phosphate buffered saline (PBS). Human sera or rabbit sera were diluted 1:200 (Gpd assays) or 1:100 (all other assays) in dilution buffer (4% milk and 0.2% Triton X-100 in PBS). The diluted sera were adsorbed overnight at 4° C. with a 0.5% (vol/vol) lysate of E. coli expressing an irrelevant Trypanosoma cruzi recombinant protein (SA85-1.1) in pRSET (Kahn, S. J. and M. Wleklinski., J. Immunol. 159:4444-4451 (1997)). This adsorption step was omitted from the sera tested for reactivity to Gpd since preliminary experiments with Gpd showed that this step had no effect on background reactivity. Samples were spun at 4° C. at 12,000×g for 10 minutes, and 50 μl of each serum were added to triplicate wells and incubated for 1 hour at room temperature. After washing, 50 μl of a 1:3,000 dilution of goat anti-human (gamma specific) F(ab′)₂ peroxidase (Sigma-Aldrich, St. Louis, Mo.) was applied and incubated at room temperature for 1 hour. Plates were developed for 30 minutes at room temperature with 100 μl of tetramethylbenzidine-H₂O₂ substrate (Kirkegaard & Perry Laboratories, Gaithersburg, Md.) per well, and the absorbance at 600 nm was measured.

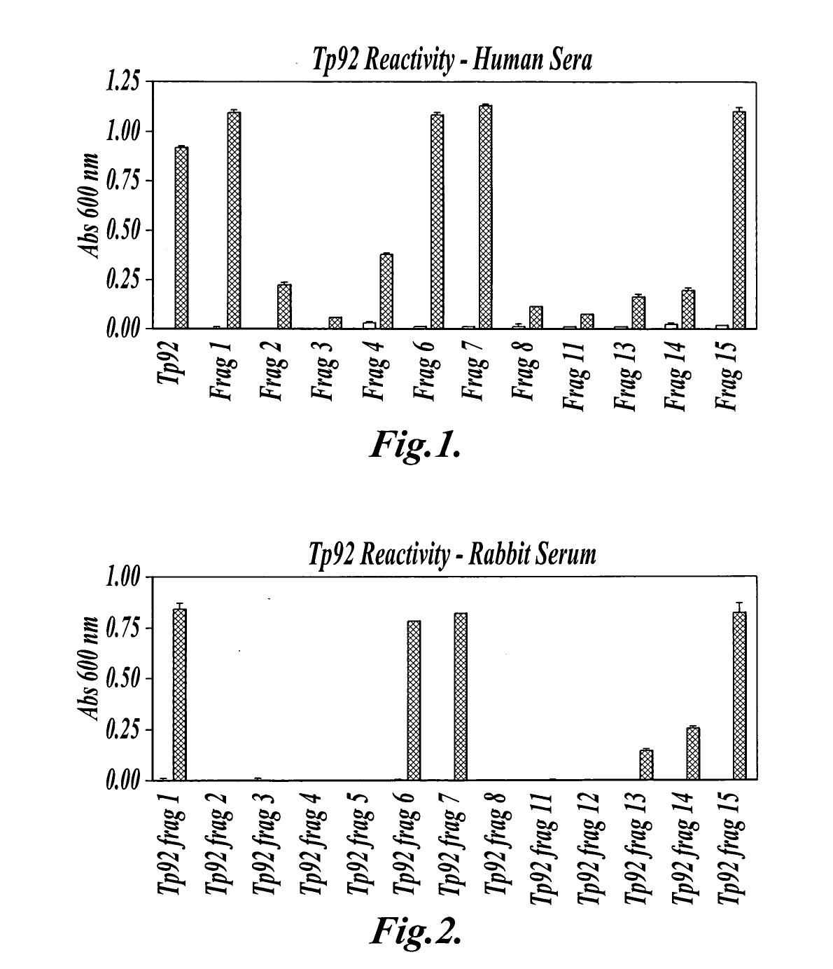

The results of these experiments are shown in FIG. 1. Additionally, similar studies were performed using serum collected from rabbits that were experimentally infected with T. pallidum. The rabbits were infected intratesticularly with 10⁵ Treponema pallidum subspecies pallidum, and serum was collected at various time points after infection. The results of these experiments are shown in FIG. 2.

These experiments showed that the focus of the antibody response against T. pallidum infection is localized to a region within recombinant fragment 7 (amino acids 26-220)(SEQ ID NO:13).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for determining whether a human subject is infected with T. pallidum, the method comprising the steps of:
(a) contacting an isolated polypeptide selected from the group consisting of a fragment of at least twelve consecutive amino acids of a Tp92 protein, a fragment of at least twelve consecutive amino acids of a Tp0453 protein, and a fragment of at least twelve consecutive amino acids of a Gpd protein, with antibodies from a human subject; and
(b) determining whether the antibodies bind to the isolated polypeptide, wherein binding of the antibodies to the isolated polypeptide indicates that the human subject is infected with T. pallidum.

2. The method of claim 1, wherein a fragment of at least twelve consecutive amino acids of the Tp92 protein is contacted with the antibodies.

3. The method of claim 2, wherein the fragment comprises amino acids 26-764 of the Tp92 protein consisting of the amino acid sequence set forth in SEQ ID NO:2.

4. The method of claim 2, wherein the fragment comprises amino acids 26-220 of the Tp92 protein consisting of the amino acid sequence set forth in SEQ ID NO:2.

5. The method of claim 1, wherein a fragment of at least twelve consecutive amino acids of the Tp0453 protein is contacted with the antibodies.

6. The method of claim 1, wherein a fragment of at least twelve consecutive amino acids of the Gpd protein is contacted with the antibodies.

7. The method of claim 1, comprising the steps of:
(a) contacting two isolated polypeptides selected from the group consisting of a fragment of at least twelve consecutive amino acids of the Tp92 protein, a fragment of at least twelve consecutive amino acids of the Tp0453 protein, and a fragment of at least twelve consecutive amino acids of the Gpd protein, with antibodies from a human subject; and
(b) determining whether the antibodies bind to the two isolated polypeptides, wherein binding of the antibodies to the two isolated polypeptides indicates that the human subject is infected with T. pallidum.

8. The method of claim 7 wherein the two members of the group consisting of a fragment of at least twelve consecutive amino acids of the Tp92 protein, a fragment of at least twelve consecutive amino acids of the Tp0453 protein, and a fragment of at least twelve consecutive amino acids of the Gpd protein are covalently linked to each other.

9. The method of claim 1, comprising the steps of:
(a) contacting an isolated fragment of at least twelve consecutive amino acids of the Tp92 protein, an isolated fragment of at least twelve consecutive amino acids of the Tp0453 protein, and an isolated fragment of at least twelve consecutive amino acids of the Gpd protein, with antibodies from a human subject; and
(b) determining whether the antibodies bind to the three isolated fragments, wherein binding of the antibodies to the three isolated fragments indicates that the human subject is infected with T. pallidum.

10. The method of claim 9 wherein the fragment of at least twelve consecutive amino acids of the Tp92 protein, the fragment of at least twelve consecutive amino acids of the Tp0453 protein, and the fragment of at least twelve consecutive amino acids of the Gpd protein are covalently linked to each other.

11. The method of claim 1, wherein the isolated polypeptide is contacted with a bodily fluid that comprises the antibodies from the human subject.

12. The method of claim 1, wherein antibody that is bound to the isolated polypeptide is detected using a labeled anti-class-specific antibody or anti-subclass specific antibody.

13. The method of claim 12, wherein the anti-class-specific antibody is selected from the group consisting of an anti-IgM antibody, an anti-IgG antibody, and an anti-IgA antibody.

14. The method of claim 12, wherein the anti-subclass-specific antibody is selected from the group consisting of an anti-IgG1 antibody, an anti-IgG2a antibody, an anti-IgG2b antibody, an anti-IgG3 antibody and an anti-IgG4 antibody.

15. The method of claim 1, wherein the isolated polypeptide is bound to a substrate.

16. A kit comprising:
(a) a substrate comprising an isolated polypeptide selected from the group consisting of a fragment of at least twelve consecutive amino acids of a Tp92 protein, a fragment of at least twelve consecutive amino acids of a Tp0453 protein, and a fragment of at least twelve consecutive amino acids of a Gpd protein;
(b) reagents for labeling antibody bound to the isolated polypeptide; and
(c) written indicia providing a user with instructions for use of the kit to determine whether a human subject is infected with T. pallidum.

17. An isolated polypeptide comprising an amino acid sequence selected from the group of amino acid sequences set forth in SEQ ID NOS:7-21.

18. An isolated polypeptide of claim 17 consisting of an amino acid sequence selected from the group of amino acid sequences set forth in SEQ ID NOS:7-21.

19. An isolated polypeptide of claim 17 comprising the amino acid sequence set forth in SEQ ID NO:13.

20. An isolated polypeptide of claim 17 consisting of the amino acid sequence set forth in SEQ ID NO:13.


SEQUENCE LISTING (21 sequences)

SEQ ID NO: 1 - DNA, 2514 bp, Treponema Pallidum
  CDS (1)..(2514)
atgctcaaaaaagccagtgccttcctaattgcaagttgttgtgtgatgtcgctggcgtgggcacaggcaaacgacaattggtacgagggaaagcctatctctgcgattagttttgaggggctcgaatatattgctcgcggccagttggacacgattttttctcaatacaagggacaaaagtggacctatgagctgtacctggagatactgcaaaaggtctatgaccttgagtacttttctgaagtttcgcctaaggcggtgcccaccgatccggagtatcagtatgtgatgctacagttcacggtaaaggagcgtccttcggtgaagggcatcaagatggtagggaacagccaaatccgcagtggggaccttttgtctaaaatcctcctgaaaaagggagacatttacaatgaagtaaagatgaaggtggaccaagagtcgctcaggcgtcattacctggaccagggctatgcggcggttaagatatcctgcgaggcaaaaactgaggcggggggcgtggtggtacagtttaccatccaggaaggtaagcagactgttgtctcgcggatacagtttaagggaaataaggcgtttaccgagtcggtgctcaagaaggtgctttccacgcaggaggcgcgttttttgaccagtggggtgttcaaggagaatgcgctggaagcggataaggcggcagtccactcatactatgcagagaggggatacattgacgcgcgggtagaaggcgtggcaaagacggttgataaaaaaactgacgccagtcgcaatctggttacgcttacgtacactgtggtggaaggtgagcagtaccgctacggcggggttaccattgtgggtaaccagatttttagcaccgaggagctgcaggcaaaaattaggctcaagcgcggggccatcatgaatatggtggcctttgagcagggctttcaggcgctggcggatgcgtattttgaaaacggatacacgtcaaattacctgaacaaagaagaacaccgggacacggcggagaaaacgctttcgtttaagatcacggtggtggagcgcgagcgcagccacgtcgagcacattatcattaagggaacgaagaatacaaaagacgaggttatcctgcgtgaaatgctgctgaaaccgggggatgtgttctctaagtcaaagtttacggatagcttgcgcaatctgttcaacctgcgctatttctcgtcgctggtgccggatgtgcggcccggctctgagcaggacctggtggacattatcctgaatgtggaggagcagtcgacggcaaacgtgcagtttggggtgacgttttctggggtgggggaggcaggcacgttcccgctttcgctcttttgtcagtgggaagaaaagaattttttgggaaaagggaatgaaatttcagtaaatgcaaccttggggtctgaggcgcagagcctgaagctcgggtatgtggagcgctggtttctgggctctccgctgacggtgggctttgactttgaacttacgcacaaaaatctctttgtgtaccgcgcgggttcatacggcaacgggctgccgcacccgtacacgagcagggagcagtgggctagttcccctgggctggcagaatcgtttcgcctcaagtattcgcgctttgagtccgccatcggcgcgcacaccgggtaccagtggtatccgcgctatgcggtcattagggtgaacgggggggtggactttcgggttgtaaagaatttttacgataaggataacaatcagcccttcgacctgaccgtaaaagagcagctgaactggaccagtatcaattcgttttggacgagcgtttcgtttgacgggcgtgactttgcgtacgacccgtccagcggctggtttttaggacagcgctgtacgttcaacgggctcgttccctttctcgaaaaagagcattcgtttcgctccgacaccaaggccgagttctacgttaccctgctcaattatccggtctctgccgtgtggaacttaaagtttgtcttggctttctacaccggtgtgtccgttcaaacgtattatggacggaggaaaagcgaaaacggaaagggcaacggggtgcggtccggcgcgctggtaatagacggcgtgctggtagggcgcgggtggagcgaagacgcaaagaaaaacaccggagacctgctgctccaccactggattgagttccgctggccgctggcgcacggcattgtgtcctttgactttttctttgatgcggcaatggtgtacaacatcgaaagtcagtccccaaacgggtcatcgtccgccagcagctccagcagcagcagtagtagtagcagtagaaccaccagctctgaaggactgtacaaaatgagctacggtccggggctgcgctttacattgccgcaatttccgttaaaattggcgttcgcaaacaccttcacgtcacccggcggcatcccaaaaacaaagaaaaattggaattttgtgttgtcgttcacggtaaataatttgtag

SEQ ID NO: 2 - protein, 837 aa, Treponema Pallidum
MLKKASAFLIASCCVMSLAWAQANDNWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFDLTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNGSSSASSSSSSSSSSSRTTSSEGLYKMSYGPGLRFTLPQFPLKLAFANTFTSPGGIPKTKKNWNFVLSFTVNNL

SEQ ID NO: 3 - DNA, 864 bp, Treponema Pallidum
  CDS (1)..(864)
ttgataaggcgtagatatcgtggttgtacgcagggagcgtggatagtaagtgttggtatgctatttgcatcgtgcacttcaggggcgtggaaggcatcagtagatccgttgggggttgtgggatctggtgcagatgtgtacctgtatttccctgtagcggggaacgagaatttgatttctcgtattatcgagaaccatgagtcaaaggcagatattaaaaaaatagtggacaggactaccgcggtatacggtgctttttttgcccgatcaaaagagtttcgtttgttcggaagcggttcgtatccatacgcctttactaatttgattttttctcgatccgatggctgggcatctacgaaaacggaacacggaatcacgtactatgaaagtgaacatacggacgtttcgattcctgcgccgcatttttcctgtgtgatttttggttcctccaaaagggagcggatgagcaaaatgctgtctcggctcgttaaccccgatcgaccgcagttaccgcctcgctttgaaaaagaatgtacgtcggaaggtacgagccagactgttgcactctatataaaaaacgggggacactttattaccaaactgttgaattttccgcagcttaatttaccacttggggcaatggaactgtacttgaccgcgcggaggaatgagtatctttacacgttgagcttgcagctggggaatgcaaagataaattttcccatacagtttttaatctctcgtgtgcttaacgcgcacattcacgtggagggggacaggttaattattgaagacggcacaatttctgctgagcgtcttgcgtcggtgatctcgtcgttgtactccaaaaagggaagttcgtaa

SEQ ID NO: 4 - protein, 287 aa, Treponema Pallidum
MIRRRYRGCTQGAWIVSVGMLFASCTSGAWKASVDPLGVVGSGADVYLYFPVAGNENLISRIIENHESKADIKKIVDRTTAVYGAFFARSKEFRLFGSGSYPYAFTNLIFSRSDGWASTKTEHGITYYESEHTDVSIPAPHFSCVIFGSSKRERMSKMLSRLVNPDRPQLPPRFEKECTSEGTSQTVALYIKNGGHFITKLLNFPQLNLPLGAMELYLTARRNEYLYTLSLQLGNAKINFPIQFLISRVLNAHIHVEGDRLIIEDGTISAERLASVISSLYSKKGSS

SEQ ID NO: 5 - DNA, 1071 bp, Treponema Pallidum
  CDS (1)..(1071)
atgcggggaacatattgtgtgacgctttgggggggggtgtttgcggcattggttgcaggctgtgcgtccgaacgtatgatagttgcgtatcggggcgctgcaggatatgtgcccgagcacacctttgcctcgaaagttcttgcttttgcacaaggagcagattacctgcagcaggatgtcgtgctttcaaaggataatcagcttatcgtagcgcaaagccatattctggataatatgactgacgtggcagaaaaatttccacgccggcagcgtgcggatgggcatttctatgtcatagattttacggtagaagaactttccctcctccgtgcaaccaatagtttctatacgcgcggtaagcgacatacgccggtgtatggccagcgctttcctctttggaagcctggttttaggctgcacacttttgaagaggagttgcagtttatccgtgggttggaacagacaaccgggaaaaagattggaatttactctgaaataaaggtgccgtggtttcatcatcaggaaggaaaagacatcgcagcgcttaccctcgctctgttgaaaaaatacggttaccaaagtcgatcggatctagtgtatgtgcaaacgtatgattttaacgagctgaagcgtatcaaacgagaacttttaccaaagtacgaaatgaacgtgaagctgattcagcgtgttgcttacacagatcaacgtgaaacacaggagaaggactcgcgtgggaaatggataaactacaattacaattggatgtttgagcccggtggtatgcagaaaatagcaaaatatgcagacggcgtgggtcctgactggaggatgctcatagagaatgaatggtcgaaggtgggcgctgttcgcctgagtccgatggtttctgcaatccaagatgcgaaattggaatgtcatgtgcacacggtacggaaagaaacactgcctagctacgcgcgcaccatggacgagatgttttccattttgttcaaacagacgggcgcaaacgtggtgctcacggattttcctgatcttggggtaaagtttctgggcaaacccgcccgctattga

SEQ ID NO: 6 - protein, 356 aa, Treponema Pallidum
MRGTYCVTLWGGVFAALVAGCASERMIVAYRGAAGYVPEHTFASKVLAFAQGADYLQQDVVLSKDNQLIVAQSHILDNMTDVAEKFPRRQRADGHFYVIDFTVEELSLLRATNSFYTRGKRHTPVYGQRFPLWKPGFRLHTFEEELQFIRGLEQTTGKKIGIYSEIKVPWFHHQEGKDIAALTLALLKKYGYQSRSDLVYVQTYDFNELKRIKRELLPKYEMNVKLIQRVAYTDQRETQEKDSRGKWINYNYNWMFEPGGMQKIAKYADGVGPDWRMLIENEWSKVGAVRLSPMVSAIQDAKLECHVHTVRKETLPSYARTMDEMFSILFKQTGANVVLTDFPDLGVKFLGKPARY

SEQ ID NO: 7 - protein, 567 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFD

SEQ ID NO: 8 - protein, 245 aa, Treponema Pallidum
LTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNGSSSASSSSSSSSSSSRTTSSEGLYKMSYGPGLRFTLPQFPLKLAFANTFTSPGGIPKTKKNWNFVLSFTVNNL

SEQ ID NO: 9 - protein, 167 aa, Treponema Pallidum
LTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIES

SEQ ID NO: 10 - protein, 89 aa, Treponema Pallidum
FDAAMVYNIESQSPNGSSSASSSSSSSSSSSRTTSSEGLYKMSYGPGLRFTLPQFPLKLAFANTFTSPGGIPKTKKNWNFVLSFTVNNL

SEQ ID NO: 11 - protein, 53 aa, Treponema Pallidum
EGLYKMSYGPGLRFTLPQFPLKLAFANTFTSPGGIPKTKKNWNFVLSFTVNNL

SEQ ID NO: 12 - protein, 739 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFDLTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNG

SEQ ID NO: 13 - protein, 195 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVF

SEQ ID NO: 14 - protein, 372 aa, Treponema Pallidum
KENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFD

SEQ ID NO: 15 - protein, 98 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGD

SEQ ID NO: 16 - protein, 97 aa, Treponema Pallidum
LLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVF

SEQ ID NO: 17 - protein, 124 aa, Treponema Pallidum
KENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKT

SEQ ID NO: 18 - protein, 112 aa, Treponema Pallidum
LSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFC

SEQ ID NO: 19 - protein, 136 aa, Treponema Pallidum
QWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFD

SEQ ID NO: 20 - protein, 372 aa, Treponema Pallidum
YFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFDLTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNGSSSASSSSSSS

SEQ ID NO: 21 - protein, 378 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLR